(19) United States
(12) Patent Application Publication
(10) Pub. No.: US 2007/0137648 A1
(43) Pub. Date: Jun. 21, 2007

(54) Intraoral Nebulizer Providing Air Curtains

(75) Inventors: W. Robert Addington, Melbourne Beach, FL (US); Stuart Miller, Indialantic, FL (US); Mary W. Briganti, Melbourne, FL (US)

Correspondence Address: ALLEN, DYER, DOPPELT, MILBRATH & GILCHRIST P.A., 1401 CITRUS CENTER 255 SOUTH ORANGE AVENUE, P.O. BOX 3791, ORLANDO, FL, 32802-3791, US

(73) Assignee: Pneumoflex Systems, LLC

(21) Appl. No.: 11/611,425

(22) Filed: Dec. 15, 2006

Related U.S. Application Data
(60) Provisional application No. 60/751,067, filed on Dec. 16, 2005.

(51) Int. Cl. A61M 16/00; A61M 11/00

(52) U.S. Cl. 128/204.25; 128/200.14

(57) ABSTRACT

An improved nebulizer places a venturi in close proximity to or inside a patient's oral cavity. One or more medicine feed lines feeds the medicine to a location proximate to a venturi. One or more air curtain conduits may be positioned near the medicine feed line and the venturi and is fed by a source of air pressure to create a curtain of fluid flow to surround at least in part the flow path of the nebulized medication. This minimizes the amount of medication lost to the oral cavity and to the trachea and permits more medication to reach a patient's lungs. Medication contained in a patients exhalant can be filtered in an exhale conduit to prevent loss to the environment and can be recaptured for reuse.

### CROSS REFERENCE TO RELATED APPLICATIONS

This application claims priority to U.S. Provisional Application Ser. No. 60/751,067, filed Dec. 16, 2005, entitled “Intraoral Nebulizer” by inventors W. Robert Addington, Stuart Miller and Mary Briganti, which is incorporated herein by reference in its entirety.

This application is also related to U.S. patent application Ser. No. 11/431,689, filed May 10, 2006, entitled “Intra-Oral nebulizer”, by inventors W. Robert Addington, Stuart Miller and Mary Briganti.

### BACKGROUND OF THE INVENTION

The invention relates to nebulizers, and, more particularly, to an intra oral nebulizer which provides one or more secondary fluid flow streams, such as air streams, forming an air curtain which helps reduce medication loss.

### DESCRIPTION OF THE PRIOR ART

Inhalation is a very old method of drug delivery. In the twentieth century it became a mainstay of respiratory care and was known as aerosol therapy. Use of inhaled epinephrine for relief of asthma was reported as early as 1929, in England. Dry powder inhalers have been utilized to administer penicillin dust to treat respiratory infections. In 1956, the first metered dosed inhaler was approved for clinical use.

The scientific basis for aerosol therapy developed relatively late, following the 1974 Sugar Loaf conference on the scientific basis of respiratory therapy.

A more complete history of the development of aerosol therapy and the modern nebulizer is described in the 2004 Phillip Kitridge Memorial Lecture entitled, “The Inhalation of Drugs: Advantages and Problems by Joseph L. Row; printed in the March 2005 issue of Respiratory Care, vol. 50, no. 3.

The typically used modern nebulizer is delivered as a kit of seven plastic pieces which are assembled prior to use to provide for delivery of the medication to a patient via inhalation. An exploded view of the seven pieces showing their relationship for assembly is given in FIG. 1. There is a mouthpiece 100 that is force fit onto one end of a T connector 110. Similarly, the other end of the T connector 110 is attached to a flex tube 120, also by force fit. The parts are such that the components can be assembled and disassembled with a simple twisting action. Nevertheless, when engaged and pressed together, the pieces form a substantially airtight seal. The bottom part of the T connector 110 is connected to a cup cover 130. That, too, is connected by pushing the cup cover onto the bottom part of the T connector in such a way that the airtight seal is formed. The cup cover 130 has a screen 135 that screens the material going into the T connector. There is a cup 150 for receiving the medicine to be nebulized. The cup also has a venturi projecting through the bottom.

In a typical use, a vial containing the medication for administration through the nebulizer is opened and poured into the cup 150 where it accumulates at the edges of the rounded bottom of the cup. The venturi is surrounded by a conical plastic piece through which it passes. The shape of the conical piece of the medicine cup 150 matches substantially the shape of the venturi cover 140. Once the medicine is poured into the cup, the venturi cover 140 is placed over the venturi and the filled medicine cup is screwed, using threaded portions on each piece, onto the cup cover 130. In this way, the medicine is held in place ready for administration.

In use, the bottom of the airline feeding the venturi in the medicine cup is attached to an air hose 160, to which is applied to a source of air pressure thus activating airflow through the venturi. By venturi action, the exhaust of the air flow through the small opening of the venturi results in a reduction in pressure on the downstream side of the airflow so that the medicine from the medicine cup is fed under positive pressure up in the interstices between the conical shape of the medicine cup and the venturi cover and is exhausted then through the screen 135 into the bottom of the T connector 110.

A patient is asked to inhale the aerosol mist provided through the cup cover screen into the airflow channel between the mouthpiece 100 and the flex tube 120. As a patient takes the mouthpiece 100 in their mouth, and inhales, air flows through the open end of the flex tube 120, through the T connector 110, picking up the aerosol medication and into the patients' air passages through the mouthpiece 100.

### PROBLEMS OF THE PRIOR ART

Table 8 of the Respiratory Care article, referred to above, page 381, lists the characteristics of an ideal aerosol inhaler as follows:

TABLE 8
Dose reliability and reproducibility
High lung-deposition efficiency (target lung deposition of 100% of
nominal dose)
Production of the fine particles ≦5 μm diameter, with correspondingly
low mass median diameter
Simple to use and handle
Short treatment time
Small size and easy to carry
Multiple-dose capability
Resistance to bacterial contamination
Durable
Cost-effective
No drug released to ambient-air
Efficient (small particle size, high lung deposition) for the specific drug
being aerosolized
Liked by patients and health care personnel

The standard nebulizer shown in FIG. 1, fails to achieve a number of these characteristics. Specifically, the nebulizer of FIG. 1 wastes medication during exhalation. Further, the particle size is often too large to reach the bottom of the lungs where the medication may be most needed. There is difficulty in estimating the dose of the drug being given to a patient and there is difficulty in reproducing that dose. There is a possibility of contamination when opening the initially sterile kit, pouring medication into the cup, and assembling the pieces for use by a patient. There is also considerable inefficiency in the medication delivery, with much of it being deposited in the throat, rather than in the lungs.

### BRIEF SUMMARY OF THE INVENTION

The invention is directed to an intra-oral or near intra-oral nebulizer that overcomes the problems of the prior art. In addition, in one embodiment of the invention, a curtain of fluid flow is positioned so as to at least partially surround the nebulizing element, e.g. a venturi, in order to facilitate direction of the flow of the fluid and the medication in such a way as to reduce medication lost to oral tissues and in a way which promotes passage of the nebulized medication toward a patient's lungs.

### BRIEF DESCRIPTION OF THE DRAWINGS

The invention will now be described with reference to the following drawings in which:

FIG. 1 is an exploded view of a nebulizer of the prior art.

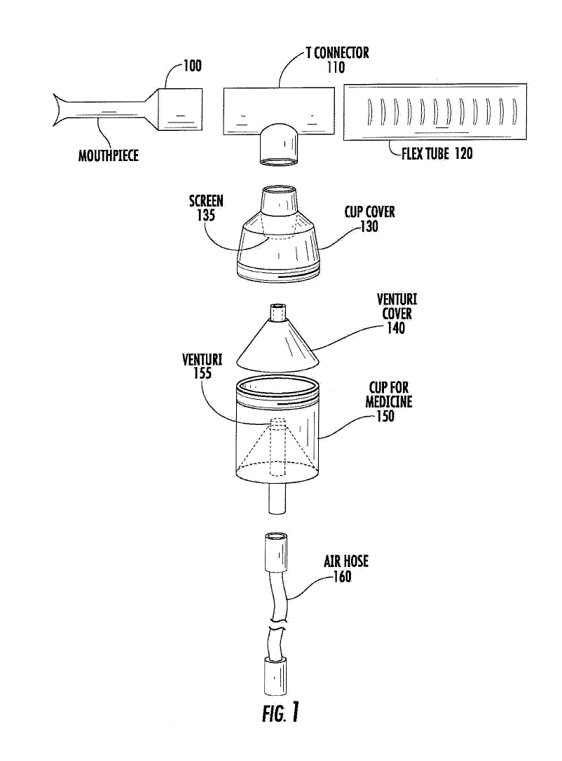

FIG. 2 is a perspective view of a nebulizer shown in pending application Ser. No. 11/431,689.

FIG. 3 is a cross section view of the nebulizer of FIG. 2.

FIG. 4 is a cross section view of the mixing end of a nebulizer in accordance with one aspect of the invention.

FIG. 5 is an end view of the nebulizer shown in FIG. 4 showing exemplary fluid curtain conduits in accordance with one aspect of the invention.

### DETAILED DESCRIPTION OF THE INVENTION

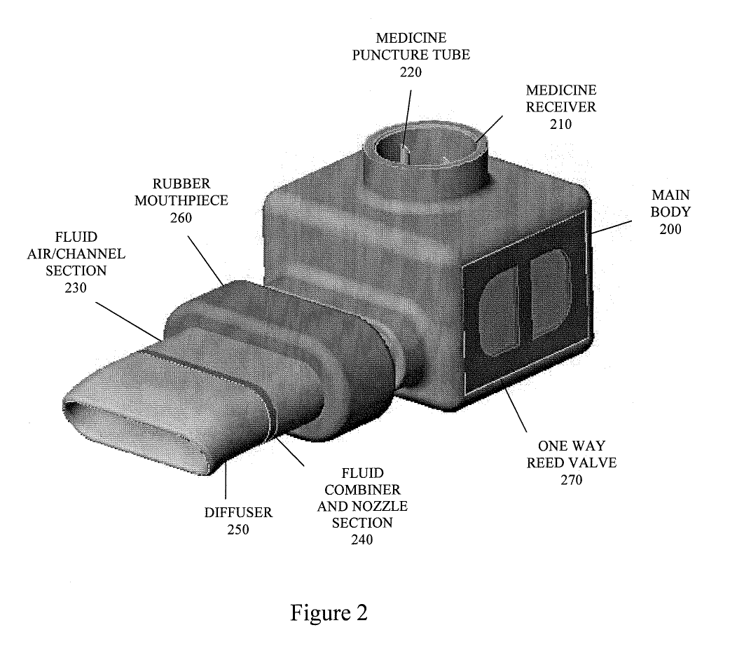

FIG. 2 is a perspective view of a nebulizer described in U.S. patent application Ser. No. 11/431,689. The nebulizer comprises a main body 200 that has a medicine receiver 210. Extending from the main body is a fluid air channel section 230. The fluid combiner and nozzle section 240 then mates the fluid air channel section 230 with the diffuser 250 as described more hereinafter. A rubber mouthpiece 260, the position of which can be adjusted, surrounds the nebulizer. The medicine receiver 210 is shaped to correspond to the shape of a medication vial or other medication container which, in this embodiment, can be punctured using the medicine puncture tubes 220 which are hollow and which permit the medication then to reach the venturi, discussed more hereinafter, utilizing, in most embodiments, a gravity feed, possibly supplemented with the venturi pressure differential.

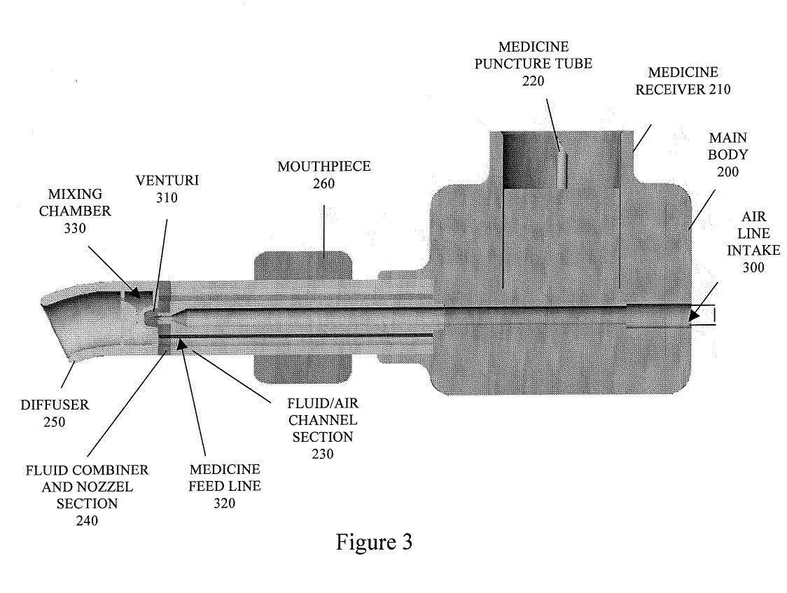

FIG. 3 is a sectional view of the nebulizer of FIG. 2, cut along the centerline of the longitudinal axis. Here one can see the path of the air from the air line 300 as it goes toward venturi 310. The medicine puncture tube 220 communicates with the medicine feed line 320 allowing the medication to flow from the medication reservoir into the medicine feed line into the mixing chamber 330 where it can be atomized by action of the venturi 310.

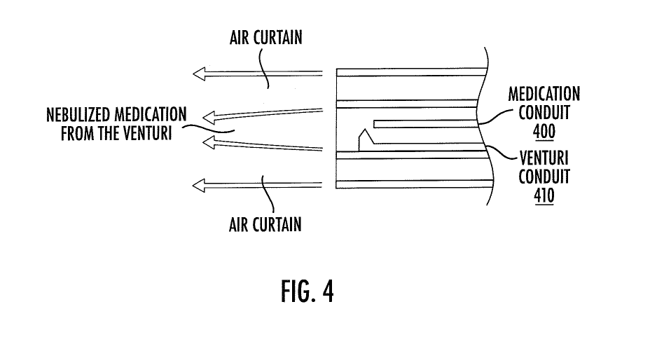

FIG. 4 is a cross section view of the mixing end of a nebulizer in accordance with one aspect of the invention. As with other nebulizers, medication conduit 400 provides a source of medication to be nebulized into appropriate sized droplets to be administered to the patient. A venturi conduit 410 feeds a fluid such as air under pressure to a venturi which is located adjacent to the end of the medication conduit, whereby a high pressure fluid flow is utilized to disperse the medication into droplets appropriately sized for delivery to the patient.

As noted above, one of the problems with administering medication to a patient in this manner is that approximately 80% of the medication inhaled by a patient undergoing treatment with a nebulizer actually gets deposited in the patients mouth and fails to reach the lungs.

As shown in FIG. 4, a stream of air or other fluid is delivered above and below the fluid stream containing the nebulized medication thus providing an air curtain or an air cushion permitting the nebulized medication to penetrate deeper into a patient's airways and minimize the loss of medication in the oral cavity. The air curtains are formed by creating a slightly positive air pressure controlled to coincide with the patients inhalation of the medication. The air curtain conduits may have one or more inlets to which is connectable a source of pneumatic or fluid pressure, such as gas, air, oxygen, or the like. The pneumatic pressure may be provided by means of a disposable single use container, or may be provided by an in-house gas system, such as the built-in oxygen line system in a hospital. A regulatory valve is preferably associated with each air curtain conduit inlet.

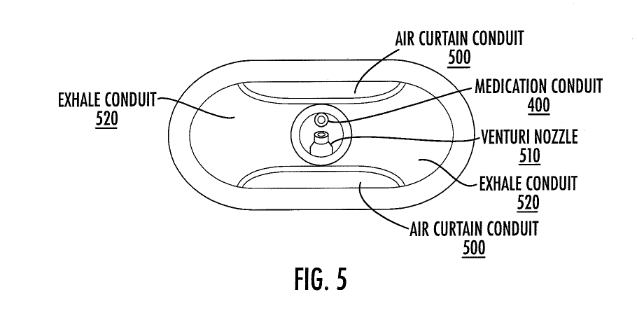

FIG. 5 is a view of the nebulizer shown in FIG. 4 showing exemplary air curtain conduits in accordance with one aspect of the invention. From this end view, one can see that there are two air curtain conduits 500; one above and one below the venturi nozzle and medication conduit. As a patient inhales, an air curtain formed under positive pressure keeps the nebulized medication at somewhat of a distance from the walls of the oral cavity and facilitates passage down the trachea. When the patient exhales, the pressure from the exhaled air or fluid (exhalant) is directed through exhale conduits 520 and vented to the surrounding environment. Each of these exhalation conduits may be disposed with a filter material effective and reduce in the amount of medication which passes out into the ambient air from the patient's exhalant. The medication contained in the exhalant may also be captured and recycled. In this manner, loss of medication is minimized.

Unless otherwise defined, technical and scientific terms used herein have the same meaning as commonly understood by one of ordinary skilled in the art to which this invention pertains. Although methods and materials similar or equivalent to those described herein can be used in the practice or testing of the present invention, suitable methods and materials are described below. In addition, the materials, methods and examples given are illustrative in nature only and not intended to be limiting. Accordingly, this invention may be embodied in many different forms and should not be construed as limited to the illustrated embodiments set forth herein. Rather, these illustrated embodiments are provided solely for exemplary purposes so that this disclosure will be thorough and complete, and will fully convey the scope of the invention to those skilled in the art.

What is claimed:

1. A nebulizer providing a curtain of air substantially surrounding air flow carrying nebulized medication.

2. The nebulizer of claim 1 in which the source of nebulized medication is designed to be positioned within an oral cavity.

3. The nebulizer of claim 1 in which the nebulizer has one or more exhale conduits for receiving exhalant from a patient being nebulized.

4. The nebulizer of claim 3 in which the exhale conduits contain a filter to prevent medications from escaping to the environment.

5. The nebulizer of claim 3 in which the exhale conduits contain a mechanism to capture and recycle medication.
